(19) United States
(12) Patent Application Publication
(10) Pub. No.: US 2026/0252172 A1
(43) Pub. Date: Aug. 27, 2026

(54) ELECTROCORTICOGRAPHIC INTERFACE DEVICE AND METHOD FOR MICRO-FABRICATING AN ELECTRODE ARRAY FOR AN ELECTROCORTICOGRAPHIC INTERFACE DEVICE

(71) Applicant: Advanced Biomedical Instrumentation Centre Limited, New Territories (CN)

(72) Inventors: Kwok Leung Chan, Pokfulam (CN); Tao Zou, New Territories (CN)

(21) Appl. No.: 19/548,364

(22) Filed: Feb. 24, 2026

Related U.S. Application Data
(60) Provisional application No. 63/763,609, filed on Feb. 26, 2025.

(51) Int. Cl. G06F 3/01; B33Y 80/00; G03F 7/40

(52) U.S. Cl. CPC G06F 3/015; B33Y 80/00; G03F 7/40

(57) ABSTRACT

An electrocorticographic interface device includes a flexible thin-film electrode array formed on a biocompatible substrate such as polyimide, the electrode array having a plurality of recording electrodes and interconnect lines patterned on the biocompatible substrate, and the flexible thin-film electrode array further includes an array of perfusion holes distributed across the biocompatible substrate.

### TECHNICAL FIELD

The present invention relates to the field of biomedical engineering and neural interfaces, and particularly, although not exclusively, to a wafer-scale fabrication and assembly method of a minimally invasive epidural electrocorticographic brain-computer interface (ECoG-BCI) device.

### BACKGROUND

Electrocorticographic brain-computer interfaces (ECoG-BCI) employ surface electrode implants to harvest neural signals from the brain's surface. These devices are an important medical instrument for recording neural activities to provide fundamental understanding in neuroscience and for clinical applications such as motor rehabilitation and speech decoding.

However, the implantation of a ECoG-BCI may require a substantial cranial opening and thus require a surgeon to perform craniotomy on the patient. This exposure of significant brain tissue to the external environment may risk complications for the patient, including brain tissue swelling, inflammation, heightening the risk of cortical damage and infection. In turn, the procedure of using a ECoG-BCI for medical diagnosis or treatment is considered a highly invasive procedure.

Furthermore, even after such an invasive procedure, conventional electrodes may not be configured to record a large cortex area with a high-density channel arrangement while simultaneously conforming to the irregular surface of the brain. In turn, obtaining the necessary measurement may be difficult, resulting in further risks to the patient when the invasive nature of the procedure would not provide the necessary benefit to the patient.

### SUMMARY OF THE INVENTION

In accordance with a first aspect of the present invention, there is provided an electrocorticographic interface device comprising:
a flexible thin-film electrode array formed on a biocompatible substrate, the electrode array having a plurality of recording electrodes and interconnect lines patterned on the biocompatible substrate.

In an embodiment of the first aspect, the flexible thin-film electrode array is arranged to structurally withstand tensile stress when implanted through a cranial opening smaller than a surface area of the flexible thin-film electrode array.

In an embodiment of the first aspect, the biocompatible substrate comprises polyimide having a thickness of approximately 7 μm.

In an embodiment of the first aspect, the polyimide provides sufficient tensile strength for the minimally invasive implantation of the electrocorticographic interface device.

In an embodiment of the first aspect, the flexible thin-film electrode array further comprises an array of perfusion holes distributed across the biocompatible substrate.

In an embodiment of the first aspect, the array of perfusion holes is arranged to allow the flow of cerebrospinal fluid to prevent fluid trapping between the recording electrodes and brain tissue.

In an embodiment of the first aspect, the array of perfusion holes is arranged to enhance the stretchability of the flexible thin-film electrode array.

In an embodiment of the first aspect, the stretchability of the flexible thin-film electrode array enables the flexible thin-film electrode array to achieve a conformal fit with a convoluted structure of a brain surface comprising sulci and gyri.

In an embodiment of the first aspect, the electrocorticographic brain-computer interface further comprises a magnetic resonance imaging (MRI) marker layer integrated within the flexible thin-film electrode array.

In an embodiment of the first aspect, the MRI marker layer is made of a ferromagnetic material such as nickel.

In an embodiment of the first aspect, the MRI marker layer is defined by a patterned nickel layer having a thickness of approximately 100 nm.

In an embodiment of the first aspect, the interconnect lines are connected to a land grid array (LGA) layout.

In an embodiment of the first aspect, the electrocorticographic brain-computer interface device further comprises a skull-mounted pedestal assembly electrically connected to the land grid array layout of the flexible thin-film electrode array.

In an embodiment of the first aspect, the skull-mounted pedestal assembly comprises a customized base surface having a curvature that is arranged to substantially match a skull structure of a specific subject.

In an embodiment of the first aspect, the skull structure is derived from a computed tomography (CT) scan of the specific subject.

In an embodiment of the first aspect, the skull-mounted pedestal assembly is fabricated from a medical-grade, MRI-compatible resin via 3D printing.

In accordance with a second aspect of the invention, there is provided a method for micro-fabricating an electrodes-array for an electrocorticographic interface device comprising the steps of:
depositing a biocompatible layer onto a wafer;
performing photolithography on the wafer to create a pattern on the biocompatible layer;
depositing a ferromagnetic layer on the biocompatible layer;
depositing a middle insulator layer on the ferromagnetic layer;
depositing a conductive layer on the biocompatible layer to produce a plurality of channels of electrodes array and interconnect lines connecting the channels of electrodes array;
depositing a top insulator layer on the electrodes array; and
detaching the electrodes array from the wafer.

In an embodiment of the second aspect, the step of depositing the biocompatible layer onto a wafer, forming the middle insulator layer and the top insulator layer is performed by spin coating polyimide onto the wafer to form the biocompatible layer as a polyimide layer or onto a respective underlying surface to form the middle insulator layer and the top insulator layer.

In an embodiment of the second aspect, the ferromagnetic layer is a nickel based layer.

In an embodiment of the second aspect, the conductive layer is formed from a Chromium layer and a gold layer.

In an embodiment of the second aspect, the Chromium layer and a gold layer have a thickness of approximately 10 nm and approximately 100 nm respectively.

In an embodiment of the second aspect, the step of detaching the electrodes array from the wafer is performed by mechanical peel-off.

In an embodiment of the second aspect, the polyimide layers are formed with an array of distributed perfusion holes.

In an embodiment of the second aspect, the method further comprises the steps of: performing soft baking, a curing process, and adjusting a thickness of the polyimide for sufficient tensile strength.

In an embodiment of the second aspect, the method further comprises the steps of: after performing photolithography on the wafer, treating the device with a reactive ion etching (RIE) process until the patterns are formed on the biocompatible layer.

### BRIEF DESCRIPTION OF THE DRAWINGS

Embodiments of the present invention will now be described, by way of example, with reference to the accompanying drawings in which:

FIG. 1 is an image illustrating an electrocorticographic interface device in accordance with one example embodiment of the present invention and a second diagram illustrating an exploded-view schematic view of the of the electrocorticographic interface device.

FIG. 2 is a schematic diagram illustrating the process steps used to create an electrodes-array for the electrocorticographic interface device of FIG. 1.

FIG. 3 shows a series of images of a wafer containing three thin-film electrode arrays with enlarged views of recording electrodes and perfusion holes; and, an image of a second wafer used to fabricate extended interconnect lines of the electrocorticographic interface device of FIG. 1.

FIG. 4 is a 3D digital model illustrating a customized pedestal and its anatomical integration with a subject's skull; and,

FIG. 5 illustrates the electrode array of the electrocorticographic interface device of FIG. 1 undergoing a bonding process used to connect the electrode array to a Printed Circuit Board (PCB).

### DETAILED DESCRIPTION OF THE PREFERRED EMBODIMENT

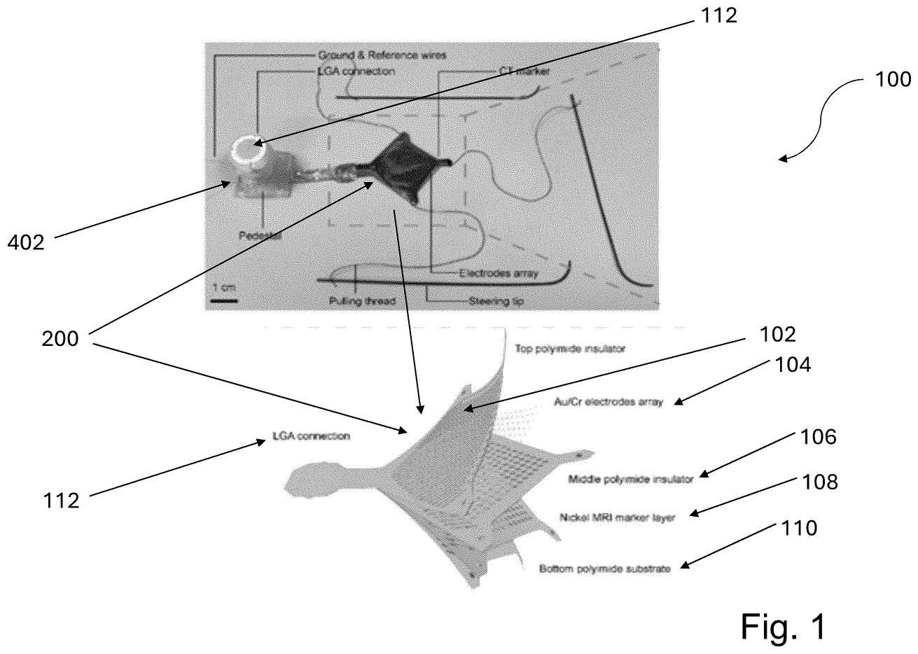

With reference to FIG. 1, there is shown an example embodiment of an electrocorticographic interface device 100 comprising: a flexible thin-film electrode array 200 formed on a biocompatible substrate, the electrode array 200 having a plurality of recording electrodes and interconnect lines patterned on the biocompatible substrate. Preferably, wherein the flexible thin-film electrode array 200 is arranged to structurally withstand tensile stress when implanted through a cranial opening smaller than a surface area of the flexible thin-film electrode array.

In this example embodiment, the invention is a large-area electrocorticographic brain-computer interface (ECoG-BCI) 100, also referred to as an electrocorticographic interface device 100 which is constructed by use of a wafer-scale fabrication and assembly method. The interface 100 is designed to record neural activities from the brain's surface while utilizing a minimal invasive implantation method that includes the use of a guidewire driven (GD) approach to minimize cerebral damage and clinical complications during implantations into a patient.

This method of implementation may be advantageous as the cranial openings required for implantations of the device 100 may be minimized in size, and thus unlike other BCI devices that require large cranial openings, this interface 100 may include a flexible, multi-layered electrode array 200 with a patient-specific, 3D-printed pedestal 402 to provide stable, long-term neural recording which may also be less invasive during the implantation of the device 100 within the patient.

Preferably, the device 100 functions by harvesting neural signals from the brain's surface via a high-density array of a plurality of (e.g. 256) recording channels, although the number of recording channels may be adjusted as necessary or as preferred. Once implanted epidurally, the flexible array 200 captures spatiotemporal neural data from the patient's brain surface. In this example, the data is then transmitted through integrated interconnect lines to a Land Grid Array (LGA) connection 112. This connection is bonded to a printed circuit board (PCB) housed within a skull-mounted pedestal 402. In turn, the pedestal 402 serves as the bridge to external equipment, such as a computer system or medical device, by providing either electrical contact points or a wireless transceiver unit to process the neural signals.

Preferably, the electrode array 200 is approximately 20 μm thick, it may be navigated into place using a steering tip and pulling threads, which would avoid the need for extensive surgery during implantation into the patient.

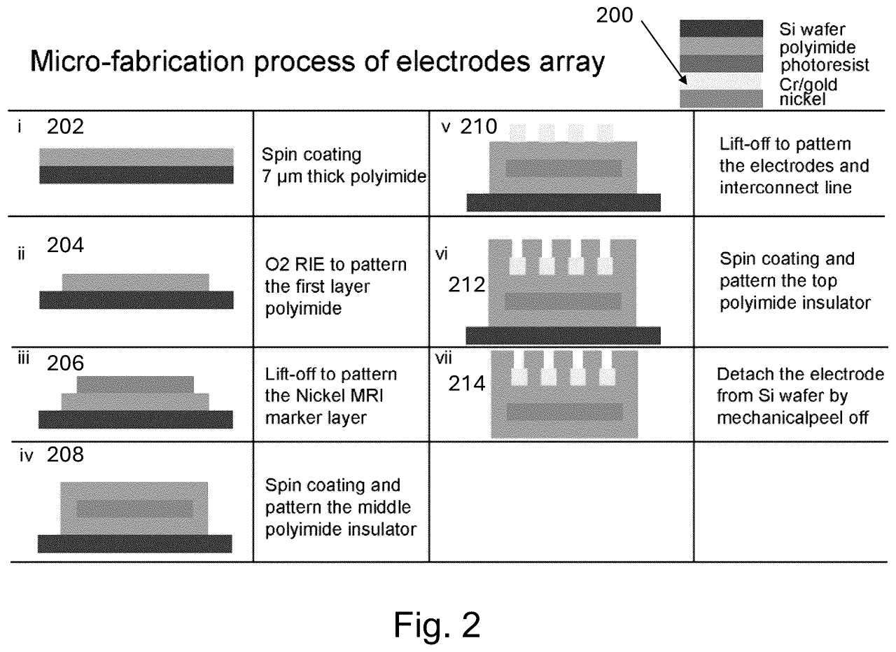

As shown in FIGS. 1 and 2, the electrocorticographic interface device 100's electrode array 200 may be fabricated in a 7-step micro-fabricated process to fabricate the thin-film array with a specialized “layered” structure. As shown in FIG. 1, the electrocorticographic interface device's electrode array 200 includes a bottom polyimide substrate 110, a Nickel MRI marker layer 108, a middle polyimide insulator 106, a gold/chromium (Au/Cr) electrode array 104, and a top polyimide insulator 102.

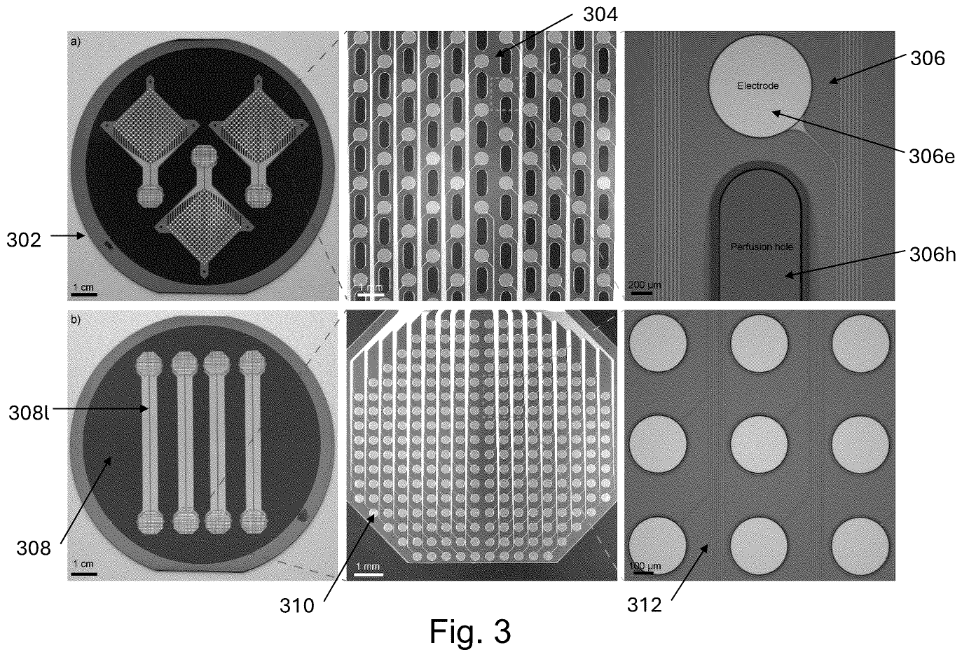

As shown, the polyimide layers 102, 106, 110 may provide the necessary biocompatibility and tensile strength and thus may be made of an appropriate thickness for example at 7 μm thick so as to provide the necessary tensile strength to withstand the mechanical stresses of guidewire implantation into the patient. Preferably, perfusion holes (see FIG. 3) are also engineered throughout the polyimide layers 102, 106, 110. These perfusion holes provide a passage or channel to allow cerebrospinal fluid (CSF) and air to pass through the device, and in turn, assisting to prevent the formation of “dead zones” and wrinkles that may reduce contact with the brain tissue or otherwise disrupt signal consistency in large-area implants.

Additionally, in some example embodiments, a customized 3D-Printed Pedestal 402 is fabricated from medical-grade resin based on a subject's CT scan data. In these examples, the pedestal's base is anatomically matched to the specific curvature of the patient's skull. This is particularly advantageous as a pedestal 402 which fits the patient's skull will minimize the presence of any gaps between the pedestal 402 and the skull when the electrocorticographic interface device 100's electrode array 200 is implanted into the patient, in turn minimizing the gaps whereby the brain tissue of the patient is exposed to an external environment.

As shown in FIG. 1, there is shown an example embodiment of a fully assembled high-density and large-area Electrocorticographic Brain-Computer Interface (ECoG-BCI) or Electrocorticographic interface device 100 designed for minimally invasive implantation. The device 100 is arranged to be connected to an external skull-mounting hardware, which is preferably the pedestal 402. As shown in this example, the device 100 is preferred to include a flexible thin-film electrode array 200 which is in turn connected via a Land Grid Array (LGA) 112. The LGA 112 also connects to the pedestal 402.

To facilitate the minimally invasive implantation of the electrode array 200 into the epidural space of the patient, the electrocorticographic interface device 100 may be implemented with a steering tip and a pulling thread, which work in tandem to allow a surgeon or medical professional to navigate the array to the target cortical site. This is particularly advantageous as smaller opening can be created in the patient's skull for the necessary craniotomy required to be performed. As shown, the LGA 112 further includes ground and reference wires extending from the assembly, which may be useful for stable neural signal acquisition.

Preferably, a CT marker is integrated into the distal end of the electrode array 200 to allow for post-operative verification of the implant's position using Computer Tomography (CT). As shown in FIG. 1, the active recording area of the electrode array 200 is approximately 2 cm×2 cm, while the total device length, including the interconnecting neck, spans several centimetres to allow for the distance between the cranial entry point and the pedestal mounting site. These dimensions may be adjusted as appropriate for a specific group of patients.

FIG. 1 also provides an exploded-view schematic of the thin-film electrode array's internal architecture. As shown, the electrode array 200 of the electrocorticographic interface device 100 includes a multi-layered “sandwich” or “layered” structure. In this example, the base layer of this electrode array includes a bottom polyimide substrate 110, which provides the mechanical foundation for the array. Above this layer is the MRI marker layer 108, which is preferably made with a ferromagnetic material such as Nickel or a material with Nickel content. This is advantageous as the ferromagnetic properties of Nickel assist to render the device visible during magnetic resonance imaging (MRI) whilst avoiding or minimizing the creation of the significant artifacts associated with other metallic components such as, but not limited to, titanium components.

This layer 108 is followed by a middle polyimide insulator 106 that separates the MRI marker 108 from the electrical components 104, which forms the functional portion of the device. These components 104 include the Au/Cr (Gold/Chromium) electrode array 104, which in this example includes a plurality (e.g. 256) high-density recording channels. Finally, the structure is sealed with a top polyimide insulator layer 102. This multi-layer arrangement is characterized by a series of perfusion holes that may be distributed openings or apertures on the device such that the openings or apertures penetrate through all polyimide layers 102, 106, 110. These holes provide a useful functional feature by providing a channel or apertures that allow cerebrospinal fluid (CSF) and air bubbles to pass through the electrode array 200 or device 100. In turn, preventing the formation of “dead zones” that would otherwise lift the electrodes away from the brain tissue. By allowing for fluid perfusion, the structure of the device 100 ensures a conformal fit, and that the device 100, which may be approximately 20 μm thick, may be able to flex and adhere closely to the complex curves of the brain's sulci and gyri region and thus maintaining high-spatiotemporal signal resolution.

With reference to FIG. 2, there is shown an example of a micro-fabrication process for the proposed electrode array 200 of the electrocorticographic interface device 100. In this example, the device 100 which includes the electrode array 200, LGA 112 and the connecting portions which connect the electrode array 200 to the LGA 112, may be fabricated or made by using a 7-step wafer-scale process. This process is used to create the thin film structure that defines the electrode array 200 of the device 100.

As shown in FIG. 2, the micro-fabrication process begins at step (i) 202 with the spin-coating of a 7-μm-thick polyimide layer onto a clean 4-inch silicon (Si) wafer. This specific thickness is preferable so as to ensure the final device to have sufficient tensile strength to withstand the mechanical stresses of the guidewire-driven implantation process. In step (ii) 204, the first layer of polyimide is patterned using O2 Reactive Ion Etching (RIE) following a photolithography process. As an example, this process may include treating the device surface with an reactive ion etching (RIE) process (Oxford Plasma Pro 100 RIE, 200 W/O2 30 sccm) until the PI pattern are complete. Step (iii) 206 involves the deposition and lift-off of the Nickel MRI marker layer which is approximately 100 nm thick. Step (iv) 208 describes the spin-coating and patterning of the middle polyimide insulator, which encapsulates the Nickel marker. In step (v) 210, the gold/chromium (Au/Cr) electrodes and interconnect lines are patterned via thermal evaporation and lift-off, creating, in this example, the 256-channel recording grid. At step (vi) 212, this step involves the application of the top polyimide insulator. The process concludes with step (vii) 214, where the completed thin film electrode array 200 is detached from the silicon wafer through a mechanical peel-off method. This systematic layering allows for the creation of a complex, high-density device that remains extremely thin (20 μm) and flexible.

With reference to FIG. 3, there is shown photographs of an example fabricated components of the electrode array 200 of the electrocorticographic interface device 100. In this example, the components are fabricated on a 4-inch silicon wafers. At 302, the photograph displays a wafer containing three thin-film electrode arrays 200. The enlarged views at 304 highlight the recording electrodes 306e and the integrated perfusion holes 306h. As shown in the highest magnification at 306, in this example embodiment, the perfusion holes 306h are positioned adjacent to the circular electrodes 306e to maximize the device's ability to release trapped fluid and air while maintaining structural integrity and optimal contact of the electrodes 306e with brain tissue. As an example, if a standard continuous film were implanted, a pocket of CSF as small as 50 microns thick could form between the electrode and the cortex, significantly attenuating the high-frequency neural signals required for accurate decoding. The perfusion holes 306h illustrated in FIG. 3 may advantageously mitigate this by providing an escape path for the fluid, ensuring direct contact. These openings 306h may also enhance the stretchability of the polyimide, facilitating a closer interface with the cortical surface of the patient's brain.

As shown in FIG. 3 at 308, the image herein (308) illustrates a second wafer used to fabricate an example of the extended interconnect lines 308l, which is arranged to connect the LGA 112 of the electrode array 200 to a PCB located in the pedestal 402. In some embodiments, as the electrocorticographic interface device may require a connection length exceeding 8 cm between the recording site and the pedestal, a length greater than what can be placed or fitted on a single 4-inch wafer alongside the electrode grid, these additional segments may be required to be manufactured separately. As shown, these lines are designed to bridge the gap between the electrode array shown in FIG. 3 at 302 and the PCB housed in the pedestal 402, ensuring that high-density signals may be transferred over a long distance without loss of integrity.

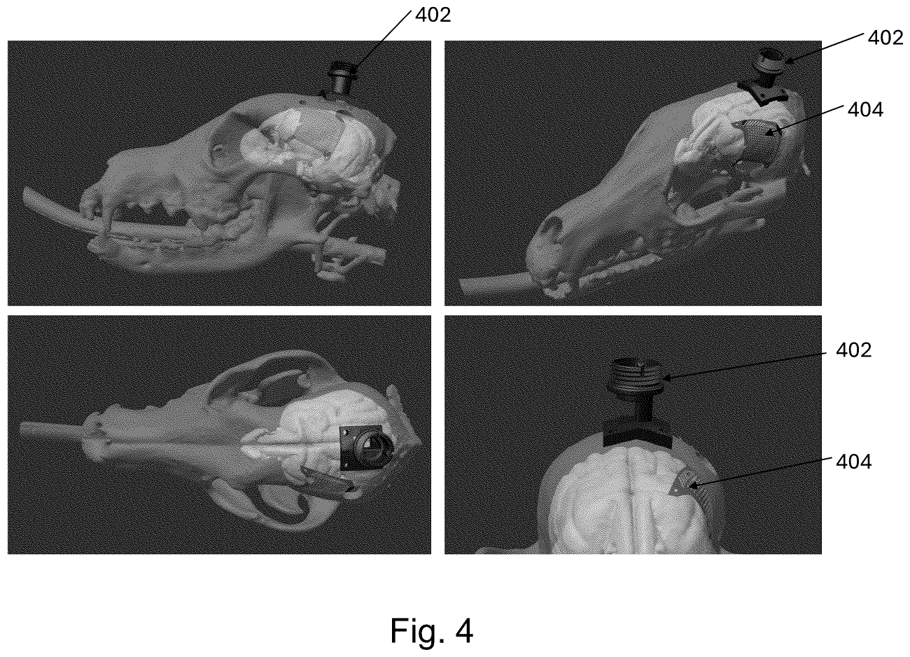

With reference to FIG. 4, there is shown a 3D digital model illustrating the customized pedestal 402 and its anatomical integration of the electrode array 404 with a subject's skull. The figure demonstrates how the pedestal 402 is designed to sit flush against the bone of the subject to ensure a seamless fit. Unlike commercial pedestals with generic flat bases, the base of this pedestal 402 is designed using CT scan data of the specific subject's skull structure, in turn, arranging that the contact surface of the pedestal 402 is closely or exactly matched to the individual's anatomy. The model shows the pedestal 402 fixed to the skull, with the electrode array 404 positioned on the brain surface. This customized fit is preferably achieved through 3D printing using medical-grade resin. This structural matching is a key advantage, as it reduces or eliminates the gaps that typically lead to fluid ingress, infection, or the eventual detachment of the implant. The subject may be given a CT or MRI scan with the data being converted into a CAD model to generate the pedestal base 402. This ensures that upon implantation, the pedestal 402 acts as a hermetic (or as close as possible) seal against the skull, preventing bacterial migration that is common with ill-fitting generic implants. Additionally, the use of medical grade resin ensures the pedestal 402 is fully MRI compatible, avoiding the imaging artifacts caused by metal pedestals. This is particularly advantageous as a patient required to use the electrocorticographic interface device 100 is likely to require ongoing monitoring and would therefore be required to undergo a pre-operative CT scans and ongoing MRI scans, and thus the reduction of any noise or artifacts would be helpful to secure an accurate and useful image for medical professionals.

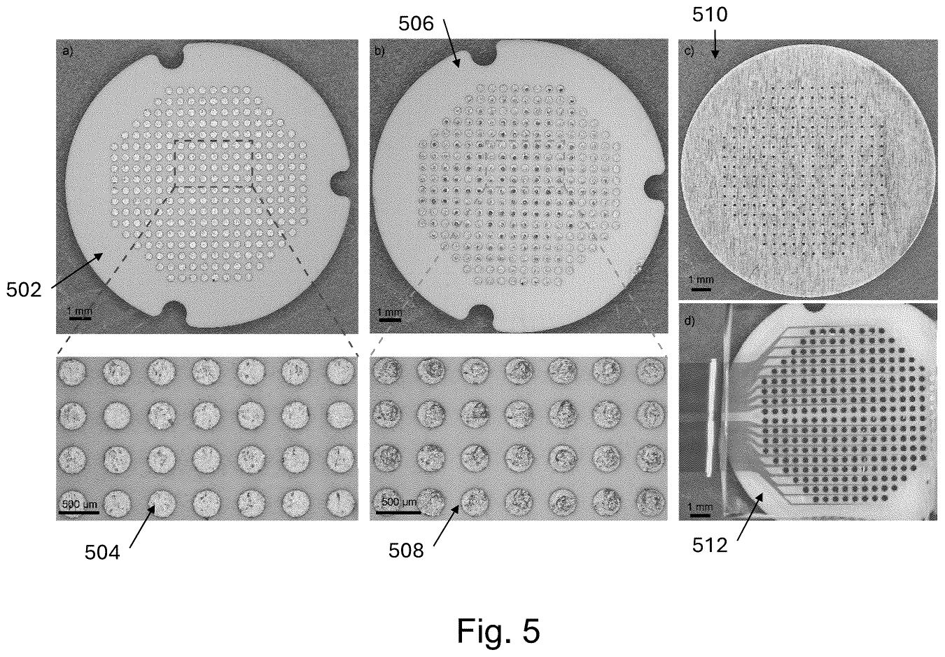

With reference to FIG. 5, there is shown the bonding process used to connect an example of the thin-film electrode array to the Printed Circuit Board (PCB). As shown in FIG. 5, the photographs 502 and 506 show a CerePort LGA PCB before and after the application of patterned silver epoxy, respectively. The silver epoxy, which is shown in 508 is used as a conductive adhesive onto the LGA electrodes 504 to create electrical paths between the electrode array's LGA connection pads 504 and the PCB 512. To achieve high-precision bonding for hundreds of channels, a 50-μm-thick metal mask 510 is used to selectively deposit the silver epoxy onto the PCB pads 512. FIG. 5 at 512 illustrates the alignment process, where a customized 6-axis micro-alignment stage and a vacuum adsorption table are used to precisely join the flexible thin-film array to the rigid PCB. This assembly method is critical for “connectorization,” which allows for a high-density, 256-channel interface that is compact enough to be housed within the small footprint of the 3D-printed pedestal.

An advantage of this example embodiment of the invention is found in its ability to provide high-density recording (e.g. 256 channels) over a large area (2 cm×2 cm) whilst minimizing the risks associated with traditional large-scale craniotomies. The structural design of the perfusion holes is also particularly advantageous as it increases the device's stretchability, whilst also enabling a conformal fit against the complex sulci and gyri of the brain by providing a passage for fluids to escape, in turn ensuring a stable and close interface for signal acquisition from the brain. Furthermore, the anatomically matched pedestal eliminates gaps between the implant and the bone, significantly reducing the risk of fluid ingress, microbial infection, and implant detachment—common failures in “flat base” pedestals. Finally, the use of resin and nickel makes the entire system MRI-compatible, allowing for functional imaging whilst reducing the heavy artifacts caused by traditional titanium or other metallic components. This combination of precision manufacturing and anatomical customization results in a device that is safer, more reliable, and more effective for BCI applications.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.
Any reference to prior art contained herein is not to be taken as an admission that the information is common general knowledge, unless otherwise indicated.
Unless otherwise defined, all technical terms used herein have the same meaning as commonly understood by one skilled in the art to which the invention belongs.
As used herein, “comprising” means including the following elements but not excluding others. “Essentially consisting of” means that the material consists of the respective element along with usual and unavoidable impurities such as side products and components usually resulting from the respective preparation or method for obtaining the material such as traces of further components or solvents. “Consisting of” means that the material solely consists of, i.e. is formed by the respective element. As used herein, the forms “a”, “an”, and “the”, are intended to include the singular and plural forms unless the context clearly indicates otherwise.

1. An electrocorticographic interface device comprising:
a flexible thin-film electrode array formed on a biocompatible substrate, the electrode array having a plurality of recording electrodes and interconnect lines patterned on the biocompatible substrate.

2. The electrocorticographic interface device according to claim 1, wherein the biocompatible substrate comprises polyimide having a thickness of approximately 7 μm.

3. The electrocorticographic interface device according to claim 1, wherein the flexible thin-film electrode array further comprises an array of perfusion holes distributed across the biocompatible substrate.

4. The electrocorticographic interface device according to claim 3, wherein the array of perfusion holes is arranged to allow the flow of cerebrospinal fluid to prevent fluid trapping between the recording electrodes and brain tissue.

5. The electrocorticographic interface device according to claim 4, wherein the array of perfusion holes is arranged to enhance the stretchability of the flexible thin-film electrode array.

6. The electrocorticographic interface device according to claim 1, further comprising a magnetic resonance imaging (MRI) marker layer integrated within the flexible thin-film electrode array.

7. The electrocorticographic interface device according to claim 6, wherein the MRI marker layer is defined by a patterned nickel layer having a thickness of approximately 100 nm.

8. The electrocorticographic interface device according to claim 1, wherein the interconnect lines are connected to a land grid array (LGA) layout.

9. The electrocorticographic interface device according to claim 8, further comprising a skull-mounted pedestal assembly electrically connected to the land grid array layout of the flexible thin-film electrode array.

10. The electrocorticographic interface device according to claim 9, wherein the skull-mounted pedestal assembly comprises a customized base surface having a curvature that is arranged to substantially matches a skull structure of a specific subject.

11. The electrocorticographic interface device according to claim 10, wherein the skull-mounted pedestal assembly is fabricated from a medical-grade, MRI-compatible resin via 3D printing.

12. A method for micro-fabricating an electrodes-array for use with an electrocorticographic interface device comprising the steps of:
depositing a biocompatible layer onto a wafer;
performing photolithography on the wafer to create a pattern on the biocompatible layer;
depositing a ferromagnetic layer on the biocompatible layer;
depositing a middle insulator layer on the ferromagnetic layer;
depositing a conductive layer on the biocompatible layer to produce a plurality of channels of electrodes array and interconnect lines connecting the channels of electrodes array;
depositing a top insulator layer on the electrodes array; and
detaching the electrodes array from the wafer.

13. A method for micro-fabricating an electrodes-array in accordance with claim 12, wherein the step of depositing the biocompatible layer onto a wafer, forming the middle insulator layer and the top insulator layer is performed by spin coating polyimide onto the wafer to form the biocompatible layer as a polyimide layer or onto a respective underlying surface to form the middle insulator layer and the top insulator layer.

14. A method for micro-fabricating an electrodes-array in accordance with claim 13, wherein the ferromagnetic layer is a nickel based layer.

15. A method for micro-fabricating an electrodes-array in accordance with claim 12, wherein the conductive layer is formed from a chromium layer and a gold layer.

16. A method for micro-fabricating an electrodes-array in accordance with claim 12, wherein the Cr layer and a gold layer have a thickness of approximately 10 nm and approximately 100 nm respectively.

17. A method for micro-fabricating an electrodes-array in accordance with claim 12, wherein the step of detaching the electrodes array from the wafer is performed by mechanical peel-off.

18. A method for micro-fabricating an electrodes-array in accordance with claim 13, wherein the polyimide layers are formed with an array of distributed perfusion holes.

19. The method of claim 13, further comprising the steps of: performing soft baking, a curing process, and adjusting a thickness of the polyimide for sufficient tensile strength.

20. The method of claim 12, further comprising the steps of: after performing photolithography on the wafer, treating the device with a reactive ion etching (RIE) process until the patterns are formed on the biocompatible layer.
